(19) United States
(12) Patent Application Publication
(10) Pub. No.: US 2007/0239269 A1
(43) Pub. Date: Oct. 11, 2007

(54) Stented Valve Having Dull Struts

(75) Inventors: Mark J. Dolan, Santa Rosa, CA (US); Jeffrey W. Allen, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/278,984

(22) Filed: Apr. 7, 2006

(51) Int. Cl. A61F 2/24; A61F 2/84

(52) U.S. Cl. 623/2.11; 623/1.26; 623/2.38

(57) ABSTRACT

A system for replacing a pulmonary valve includes a conduit having a lumen, a delivery catheter and a replacement valve device disposed on the delivery catheter. The replacement valve device includes a prosthetic valve connected to a valve support region of an expandable support structure. The valve support region includes a plurality of protective struts disposed between a first stent region and a second stent region. A method for replacing a pulmonary valve includes implanting a conduit and delivering a replacement valve device to the conduit. The replacement valve device includes a valve connected to a valve support region that includes a plurality of protective struts. The method also includes deploying the prosthetic valve device from a delivery catheter into the lumen, positioning the prosthetic valve device within the conduit lumen and expanding the prosthetic valve device into contact with the inner wall of the conduit.

### TECHNICAL FIELD

This invention relates generally to medical devices for treating cardiac valve abnormalities, and particularly to a pulmonary valve replacement system and method of employing the same.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

The pulmonary valve regulates blood flow between the right ventricle and the pulmonary artery, controlling blood flow between the heart and the lungs. Pulmonary valve stenosis is frequently due to a narrowing of the pulmonary valve or the pulmonary artery distal to the valve. This narrowing causes the right side of the heart to exert more pressure to provide sufficient flow to the lungs. Over time, the right ventricle enlarges, which leads to congestive heart failure (CHF). In severe cases, the CHF results in clinical symptoms including shortness of breath, fatigue, chest pain, fainting, heart murmur, and in babies, poor weight gain. Pulmonary valve stenosis most commonly results from a congenital defect, and is present at birth, but is also associated with rheumatic fever, endocarditis, and other conditions that cause damage to or scarring of the pulmonary valve. Valve replacement may be required in severe cases to restore cardiac function.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques. As a consequence, replacement of the pulmonary valve has become a treatment option for pulmonary valve stenosis.

The most severe consequences of pulmonary valve stenosis occur in infants and young children when the condition results from a congenital defect. Frequently, the pulmonary valve must be replaced with a prosthetic valve when the child is young, usually less than five years of age. However, as the child grows, the valve can become too small to accommodate the blood flow to the lungs that is needed to meet the increasing energy demands of the growing child, and it may then need to be replaced with a larger valve. Alternatively, in a patient of any age, the implanted valve may fail to function properly due to calcium buildup and have to be replaced. In either case, repeated surgical or transvenous procedures are required.

To address the need for pulmonary valve replacement, various implantable pulmonary valve prostheses, delivery devices and surgical techniques have been developed and are presently in use. One such prosthesis is a bioprosthetic, valved conduit comprising a glutaraldehyde treated bovine jugular vein containing a natural, trileaflet venous valve, and sinus. A similar device is composed of a porcine aortic valve sutured into the center of a woven fabric conduit. A common conduit used in valve replacement procedures is a homograft, which is a vessel harvested from a cadaver. Valve replacement using either of these devices requires thoracotomy and cardiopulmonary bypass.

When the valve in the prostheses must be replaced, for the reasons described above or other reasons, an additional surgery is required. Because many patients undergo their first procedure at a very young age, they often undergo numerous procedures by the time they reach adulthood. These surgical replacement procedures are physically and emotionally taxing, and a number of patients choose to forgo further procedures after they are old enough to make their own medical decisions.

Recently, implantable stented valves have been developed that can be delivered transvenously using a catheter-based delivery system. These stented valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The stented valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at the delivery site, where the stent portion is expanded against the wall of a previously implanted prostheses or a native vessel to hold the valve firmly in place.

One embodiment of a stented valve is disclosed in U.S. Pat. No. 5,957,949 titled “Percutaneous Placement Valve Stent” to Leonhardt, et al, the contents of which are incorporated herein by reference.

A problem with delivering stented valves, however, is the potential for damaging the valve when the stented valve is crimped onto the delivery device and when the stented valve is expanded at the treatment site. Of particular concern is damage to the valve and the stent graft that may be caused by the edges of squared corners on the struts during crimping and expansion. The squared edges of the stent struts can also cause damage to the valve leaflets, and other valve structure, after the valve is implanted into a patient's vascular system.

It would be desirable, therefore, to provide an implantable heart valve that can readily be replaced, and would overcome the limitations and disadvantages inherent in the devices described above.

### SUMMARY OF THE INVENTION

It is an object of the present invention to provide a vascular valve replacement system having at least a delivery catheter and a replacement valve device disposed on the delivery catheter. The replacement valve device includes a prosthetic valve connected to a valve support region of an expandable support structure. The valve support region includes a plurality of protective struts disposed between a first stent region and a second stent region.

The system and the prosthetic valve will be described herein as being used for replacing a pulmonary valve. The pulmonary valve is also known to those having skill in the art as the “pulmonic valve” and as used herein, those terms shall be considered to mean the same thing.

Thus, one aspect of the present invention provides a pulmonary valve replacement system. The system comprises a conduit having a lumen, a delivery catheter and a replacement valve device disposed on the delivery catheter. The replacement valve device includes a prosthetic valve connected to a valve support region of an expandable support structure. The valve support region includes a plurality of protective struts disposed between a first stent region and a second stent region.

Another aspect of the invention provides a pulmonary valve replacement system comprising a conduit having an interior wall forming a lumen and a replacement valve device. The replacement valve device includes a prosthetic valve connected to a valve support region of an expandable support structure and the valve support region includes a plurality of protective struts disposed between a first stent region and a second stent region.

Another aspect of the invention provides a method for replacing a pulmonary valve. The method comprises implanting a conduit into a target region of a vessel and delivering a replacement valve device to the lumen of the conduit. The replacement valve device includes a valve connected to a valve support region of an expandable support structure, and the valve support region includes a plurality of protective struts disposed between a first stent region and a second stent region of the expandable support structure. The method also includes deploying the prosthetic valve device from a delivery catheter into the lumen, positioning the prosthetic valve device within the conduit lumen and expanding the prosthetic valve device into contact with the inner wall of the conduit.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic interior view of a human heart showing the functioning of the four heart valves;

FIG. 2A is a schematic view showing the placement of a pulmonary conduit, as is known in the prior art;

FIG. 2B is a schematic view showing attachment of a pulmonary conduit to the pulmonary artery, as is known in the prior art;

FIG. 2C is a schematic view showing attachment of a pulmonary conduit to the heart, as is known in the prior art;

FIG. 3 is a schematic view of one embodiment of a prosthetic valve device, in accordance with the present invention;

FIGS. 4 to 6 are cross-sectional views of exemplary protective struts for use in the prosthetic valve device illustrated in FIG. 3;

FIG. 7 is a cross-sectional view of another exemplary protective strut for use in the prosthetic valve device illustrated in FIG. 3;

FIG. 8 is a schematic view of another embodiment of a prosthetic valve device, in accordance with the present invention; and

FIG. 9. is a flow diagram of a method of treating right ventricular outflow tract abnormalities by replacing a pulmonary valve, in accordance with the present invention.

### DETAILED DESCRIPTION

The invention will now be described by reference to the drawings wherein like numbers refer to like structures.

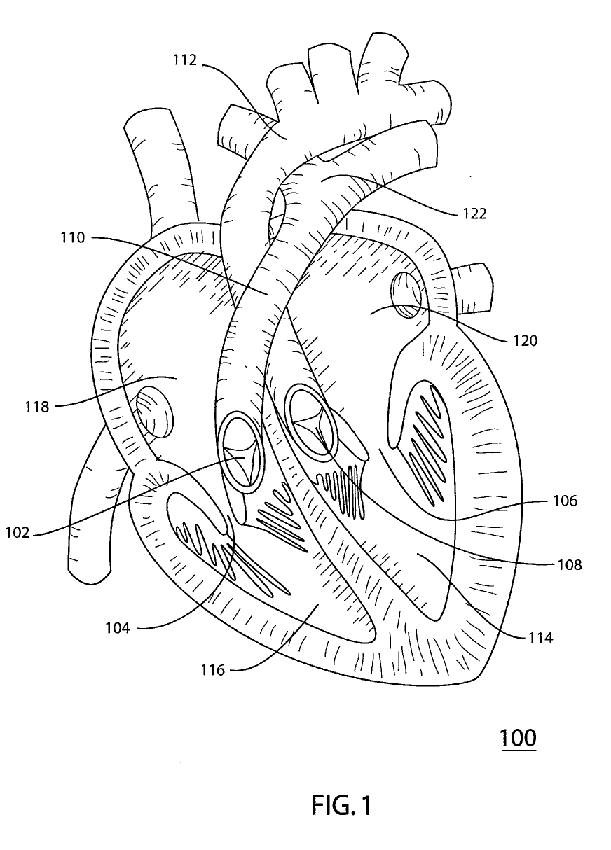

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Aortic valve 108 is situated at the junction between aorta 112 and left ventricle 114 and facilitates blood flow from heart 100, through aorta 112 to the peripheral circulation.

Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other. During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

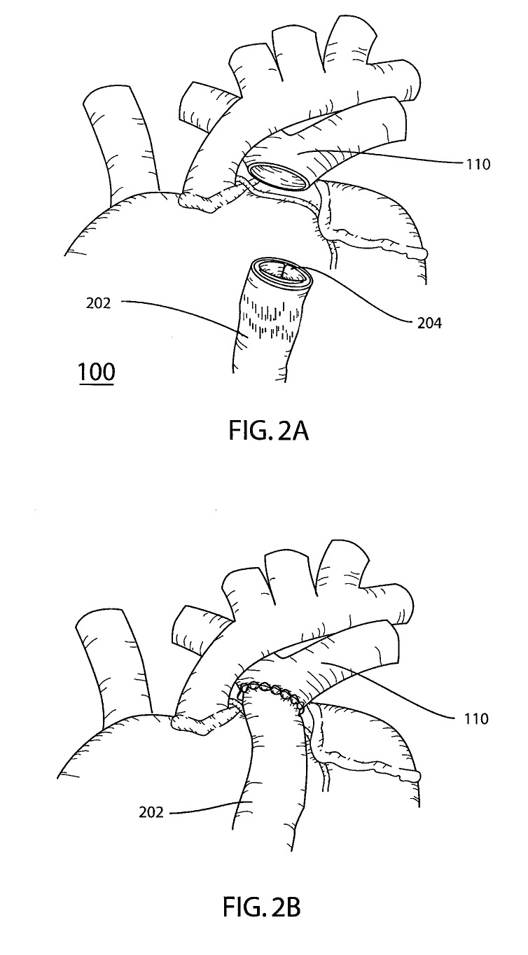

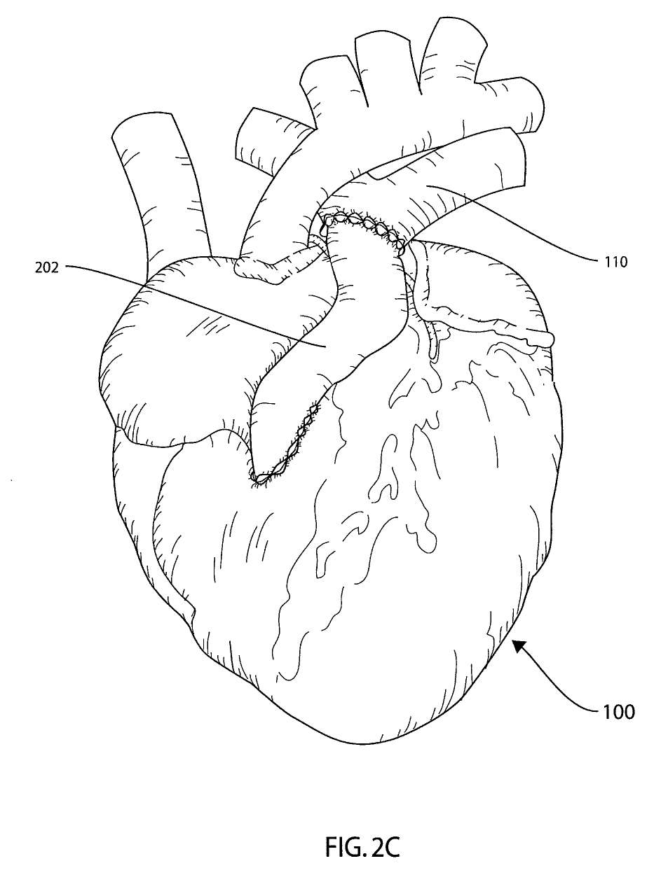

The right ventricular outflow tract is the segment of pulmonary artery 110 that includes pulmonary valve 102 and extends to branch point 122, where pulmonary artery 110 forms left and right branches that carry blood to the left and right lungs respectively. A defective pulmonary valve or other abnormalities of the pulmonary artery that impede blood flow from the heart to the lungs sometimes require surgical repair or replacement of the right ventricular outflow tract with prosthetic conduit 202, as shown in FIG. 2A-C.

Such conduits comprise tubular structures of biocompatible materials, with a hemocompatible interior surface. Examples of appropriate biocompatible materials include polytetrafluoroethylene (PTFE), woven polyester fibers such as Dacron® fibers (E.I. Du Pont De Nemours & Co., Inc.), and xenograft vein cross linked with glutaraldehyde. One common conduit is a homograft, which is a vessel harvested from a cadaver and treated for implantation into a recipient's body. These conduits may contain a valve at a fixed position within the interior lumen of the conduit that functions as a replacement pulmonary valve. One such conduit 202 comprises a bovine jugular vein with a trileaflet venous valve preserved in buffered glutaraldehyde. Other valves are made of synthetic materials and are attached to the wall of the lumen of the conduit. The conduits may also include materials having a high X-ray attenuation coefficient (radiopaque materials) that are woven into or otherwise attached to the conduit, so that it can be easily located and identified.

As shown in FIGS. 2A and 2B, conduit 202, which houses valve 204 within its inner lumen, is installed within a patient by sewing the distal end of conduit 202 to pulmonary artery 110, and, as shown in FIG. 2C, attaching the proximal end of conduit 202 to heart 100 so that the lumen of conduit 202 connects to right ventricle 116.

Over time, implanted prosthetic conduits and valves are frequently subject to calcification, causing the affected conduit or valve to lose flexibility, become misshapen, and lose the ability to function effectively. Additional problems are encountered when prosthetic valves are implanted in young children. As the child grows, the valve will ultimately be too small to handle the increased volume of blood flowing from the heart to the lungs. In either case, the valve needs to be replaced.

The current invention discloses devices and methods for percutaneous catheter based placement of stented valves for regulating blood flow through a pulmonary artery. In a preferred embodiment, the valves are attached to an expandable support structure and they are placed in a valved conduit that is been attached to the pulmonary artery, and that is in fluid communication with the right ventricle of a heart. The support structure can be expanded such that any pre-existing valve in the conduit is not disturbed, or it can be expanded such that any pre-existing valve is pinned between the support structure and the interior wall of the conduit.

The delivery catheter carrying the stented valve is passed through the venous system and into a patient's right ventricle. This may be accomplished by inserting the delivery catheter into either the jugular vein or the subclavian vein and passing it through superior vena cava into right atrium. The catheter is then passed through the tricuspid valve, into right ventricle, and out of the ventricle into the conduit. Alternatively, the catheter may be inserted into the femoral vein and passed through the common iliac vein and the inferior vena cava into the right atrium, then through the tricuspid valve, into the right ventricle and out into the conduit. The catheters used for the procedures described herein may include radiopaque markers as are known in the art, and the procedure may be visualized using fluoroscopy, echocardiography, ultrasound, or other suitable means of visualization.

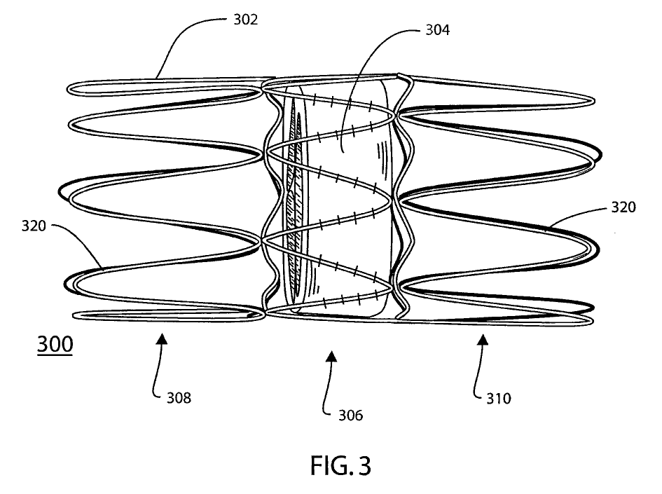

FIG. 3 is a side view of one embodiment of a replacement valve device 300, in accordance with the present invention. Replacement valve 300 is suitable for use in either a prosthetic conduit such as conduit 202, in the pulmonary artery 110, or to replace other valves in the cardiac structure. Replacement valve 300 may also be referred to herein as stented valve 300. Prosthetic valve 304 is situated within the lumen of expandable tubular support structure 302. In one embodiment of the invention, support structure 302 is a stent made of a flexible, biocompatible material that has “shape memory”, such as nitinol. In one embodiment, prosthetic valve 304 comprises three leaflets of a flexible material.

Support structure 302 comprises a first stent region 308, a second stent region 310 and a valve support region 306 disposed between the first stent region 308 and the second stent region 31 0. Valve support region 306 comprises a stent framework composed of a plurality of protective struts 312. The stent can be made by any means known in the art, including chemical etching, and laser cutting a tube of material. An example of a suitable stent for use in a system for replacing cardiac valves is shown in the U.S. Patent Application having the publication No. 2005/0203605, titled “RADIALLY CRUSH RESISTANT STENT,” for Dolan, the contents of which are incorporated herein by reference.

Embodiments of the current invention have stents with struts that are dulled or otherwise broadened such that the edges will not easily cut into the delicate valve structure. In one embodiment, protective struts 312 have a rounded transverse cross section to prevent the struts from cutting or otherwise damaging the valve or graft material on the stent when it is crimped into a delivery configuration or when it is expanded.

One method for creating rounded edges on the struts of a stent is electropolishing, where an electric current is run through the stent in a conductive aqueous bath made of salts that are similar to the base metal being polished. A cathode is positioned either outside the stent diameter or inside the stent diameter. As the electricity jumps from the stent (acting as an anode) to the cathode, material is removed. Material preferentially comes off of the peaks, which are also the square edges of the stent. As the material is removed from the square edge, it becomes rounded or dull. Adjusting the position of the cathode can adjust how the material is removed from the peaks (i.e., more material is removed from the inside peaks if the cathode is inside the stent diameter).

Another method for rounding off the square edges of stent struts is tumbling, wherein the stent is first expanded to a workable diameter. The stent is then placed in a mixture of media that typically includes silicon carbide and water with silicon carbide impregnated alumina or plastic. The mixture is placed in drum that is rotated at a speed that will maximize tumbling action. The action of the media rubbing against the stent will remove the square cut edges from the strut. The way the material is removed from the stent can be adjusted based on how far the stent is expanded before tumbling and how much water is added to the tumbling mixture. This process is described in greater detail in the international patent application No. PCT/US03/41649, titled “METHOD FOR MANUFACTURING AN ENDOVASCULAR SUPPORT DEVICE,” the contents of which are incorporated herein by reference.

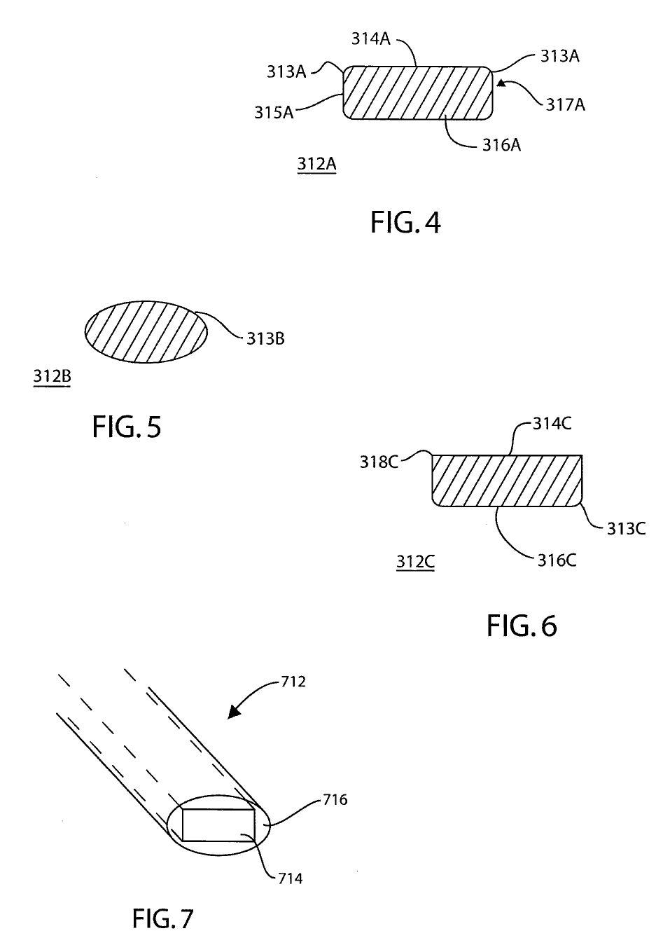

The current invention provides valve support structures having transverse cross sections (a cross section taken at a right angle to the long axis of a member) with rounded edges so that the cross sections do not have four right angle corners like a strut having a square or rectangular cross section would. FIGS. 4 to 6 illustrate various embodiments of strut 312 for use in valve support region 306. FIG. 4 illustrates a protective strut 312A. In this embodiment, protective strut 312A has a transverse cross section with rounded edges 313A on the outer surface 314A and on the inner surface 316A that contacts the valve. The rounded edges, exist as arched transitions between the flat planes 314A-317A.

FIG. 5 illustrates a protective strut 312B. In this embodiment, protective strut 312B has an oval shaped transverse cross section with rounded ends 313B. In one embodiment of the invention having struts with an oval shaped transverse cross section, the interior and exterior surfaces are essentially flat, and in another they are gently rounded. In another embodiment, the transverse cross section of the struts is circular or round in shape. FIG. 6 illustrates a protective strut 312C. In this embodiment, protective strut 312C has an elongate cross section with rounded edges 313C on the inner surface 316C that contacts the valve and squared edges 318C on the outer surface 314C. In one preferred embodiment of the invention, the stent members in the first and second stent regions have transverse cross sections with the same shape as the transverse cross section of the protective struts.

First stent region 308 and second stent region 310 each comprise a stent framework composed of a plurality of struts 320. In one embodiment, struts 320 have a cross section similar to, or the same as, the cross section of protective strut 312. In another embodiment, struts 320 have a square or rectangular cross section. Those with skill in the art will recognize that the valve support region with the protective struts may be disposed between a variety of stent regions other than those described without departing from the scope of the present invention.

The stent framework of first stent region 308 and second stent region 310 may be composed of self-expanding material and manufactured from, for example, a nickel titanium alloy and/or other alloy(s) that exhibit superelastic behavior. Other suitable materials for first stent region 308 and second stent region 310 include, but are not limited to, ceramic, tantalum, stainless steel, titanium ASTM F63-83 Grade 1, niobium, high carat gold K 19-24, platinum iridium alloys, nitinol, and cobalt based alloys. Furthermore, the stent framework material may include polymeric biocompatible materials recognized in the art for such devices.

The support structure 302 and/or stent framework may also include materials having a high X-ray attenuation coefficient (radiopaque materials) so that the replacement valve device can be easily located and identified. Examples of suitable materials include, but are not limited to, gold, silver, tantalum oxide, tantalum, platinum, platinum/iridium alloy, tungsten and combinations thereof. The radiopaque material may be visualized by fluoroscopy, IVUS, and other methods known in the art.

FIG. 7 illustrates a cross-sectional view of another embodiment of a protective strut 712 suitable for use in the valve support region 306 illustrated in FIG. 3. Protective strut 712 comprises a strut member 714 having a protective layer 716 surrounding the strut member to provide a generally rounded or oval cross section. Protective layer 712 encloses the strut member 712 in such a manner as to cover the corners and edges of the strut member thereby reducing or eliminating contact of the prosthetic valve with the edges of the strut that may damage the valve during crimping and expansion of the stented valve.

In one embodiment, protective layer 716 comprises a biodegradable coating that erodes over a period of time after implantation of the stented valve within the vessel or conduit. Examples of biodegradable polymers suitable for use include but are not limited to bioabsorbable polymers such polyphosphate ester, polyhydroxybutyrate valerate, and poly (L-lactic acid) to form a uniform coating on the exterior surface of strut members 714 that erodes over a defined period of time.

In one embodiment, the biodegradable polymer includes a therapeutic agent that is released as the biodegradable polymer erodes. The therapeutic agent comprises one or more drugs, polymers, a component thereof, a combination thereof, and the like. For example, the therapeutic agent can include a mixture of a drug and a polymer as known in the art. Some exemplary drug classes that may be included are antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific examples of drugs include abciximab, angiopeptin, colchicine, eptifibatide, heparin, hirudin, lovastatin, methotrexate, streptokinase, taxol, ticlopidine, tissue plasminogen activator, trapidil, urokinase, and growth factors VEGF, TGF-beta, IGF, PDGF, and FGF.

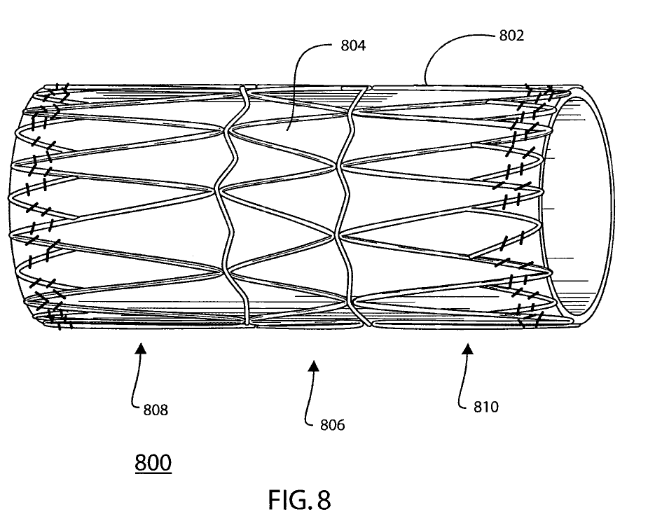

FIG. 8 is a side view of another embodiment of a replacement valve device 800, in accordance with the present invention. Replacement valve 800 is suitable for use in either a prosthetic conduit such as conduit 202, in the pulmonary artery 110, or to replace other valves in the cardiac structure. Replacement valve 800 may also be referred to herein as stented valve 800. Prosthetic valve 804 is situated within the lumen of expandable tubular support structure 802. In one embodiment of the invention, support structure 802 is a stent made of a flexible, biocompatible material that has “shape memory”, such as nitinol. In one embodiment, prosthetic valve 804 comprises three leaflets of a flexible material.

Support structure 802 comprises a first stent region 808, a second stent region 810 and a valve support region 806 disposed between the first stent region 808 and the second stent region 810. In this embodiment, valve support region 806, first stent region 808 and second stent region 810 comprise a stent framework composed of a plurality of protective struts 812. The stent can be made by any means known in the art, including chemical etching, and laser cutting a tube of material.

Protective struts 812 are dulled or otherwise broadened such that the edges will not easily cut into the delicate valve structure. In one embodiment, protective struts 812 have a rounded transverse cross section to prevent the struts from cutting or otherwise damaging the valve or graft material on the stent when it is crimped into a delivery configuration or when it is expanded. The method for creating rounded edges on the protective struts 812 of support structure 802 may be the same or similar to the methods described above for protective struts 312. The protective struts 812 of support structure 802 have transverse cross sections the same as or similar to those described above and illustrated in FIGS. 4-6.

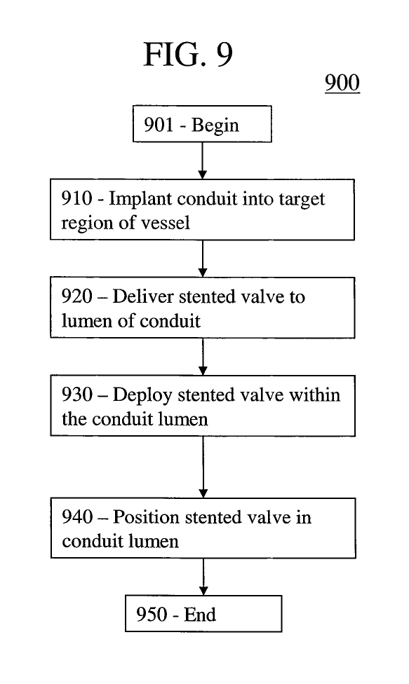

FIG. 9 is a flowchart illustrating method 900 for treating right ventricular outflow tract abnormalities by replacing a pulmonary valve, in accordance with the present invention. Method 900 starts at 901. Method 900 begins with the implantation of a conduit into the target region of a vessel. In one embodiment, and as illustrated in FIGS. 1-2C, the conduit is implanted to replace a pulmonary artery (Block 910).

Method 900 continues with the insertion and positioning of a distal end of a delivery tube at the treatment site (Block 920). The distal portion of a delivery catheter is inserted into the vascular system of the patient, and is then passed through the venous system and into a patient's right ventricle 116. This may be accomplished by inserting delivery catheter into either the jugular vein or the subclavian vein, and passing it through the superior vena cava into right atrium 118. The catheter is then passed through tricuspid valve 104, into right ventricle 116, and out of the ventricle into either conduit 202 or the pulmonary artery. Alternatively, delivery catheter may be inserted into the femoral vein and passed through the common iliac vein and the inferior vena cava into right atrium 118, then through tricuspid valve 104, into right ventricle 116, and out into conduit 202.

The catheters used for the procedures described herein may include radiopaque markers as are known in the art, and the procedure may be visualized using fluoroscopy, echocardiography, ultrasound, or other suitable means of visualization. The distal portion of delivery catheter is then positioned at the treatment site within conduit 202.

Next, stented valve 300 is deployed from the delivery catheter (Block 930), and expanded into position within conduit 202 (Block 940). Stented valve 300 is delivered to the conduit 202 or vessel in a collapsed state. Stented valve 300 expands upon deployment from the catheter. Stented valve 300 may include radiopaque markers to aid in the visualization of the stented valve during implantation. Method 900 ends at Block 950.

While the invention has been described with reference to particular embodiments, it will be understood by one skilled in the art that variations and modifications may be made in form and detail without departing from the spirit and scope of the invention.

What is claimed is:

1. A vascular valve replacement system, the system comprising:
a delivery catheter;
a replacement valve device disposed on the delivery catheter;
the replacement valve device including a prosthetic valve connected to a valve support region of an expandable support structure;
the valve support region having a plurality of struts disposed between a first stent region and a second stent region; and
each strut having a plurality of rounded edges such that the transverse cross-sectional shape of the strut does not have four right angle corners.

2. The system of claim 1 wherein the protective struts include rounded edges adjacent an inner surface of the protective struts and squared edges adjacent an outer surface of the protective strut.

3. The system of claim 1 wherein the protective struts comprises a strut member and a protective layer surrounding the strut member.

4. The system of claim 3 wherein the protective layer comprises a biodegradable coating.

5. The system of claim 4 wherein the biodegradable coating comprises a biodegradable polymer.

6. The system of claim 5 wherein the biodegradable polymer comprises a polymer selected from a group consisting of polyphosphate ester, polyhydroxybutyrate valerate, and poly (L-lactic acid).

7. The system of claim 4 wherein biodegradable coating includes a therapeutic agent.

8. The system of claim 1 wherein the system further comprises a conduit having a lumen;

9. A pulmonary valve replacement system, the system comprising:
a conduit having an interior wall forming a lumen;
a replacement valve device, the replacement valve device including a prosthetic valve connected to a valve support region of an expandable support structure,
wherein the valve support region includes a plurality of protective struts disposed between a first stent region and a second stent region.

10. The system of claim 9 wherein the protective struts include a plurality of rounded edges.

11. The system of claim 10 wherein the protective struts include rounded edges adjacent an inner surface of the protective struts and squared edges adjacent an outer surface of the protective strut.

12. The system of claim 9 wherein the protective struts comprises a strut member and a protective layer surrounding the strut member.

13. The system of claim 12 wherein the protective layer comprises a biodegradable coating.

14. The system of claim 13 wherein the biodegradable coating comprises a biodegradable polymer.

15. The system of claim 14 wherein the biodegradable polymer comprises a polymer selected from a group consisting of polyphosphate ester, polyhydroxybutyrate valerate, and poly (L-lactic acid).

16. The system of claim 13 wherein biodegradable coating includes a therapeutic agent.

17. A method for replacing a pulmonary valve, the method comprising:
implanting a conduit into a target region of a vascular system, the conduit having an inner wall defining a lumen;
delivering a replacement valve device to the lumen of the conduit, the replacement valve device including a valve connected to a valve support region of an expandable support structure, the valve support region including a plurality of protective struts disposed between a first stent region and a second stent region of the expandable support structure;
deploying the prosthetic valve device from a delivery catheter into the lumen;
positioning the prosthetic valve device within the conduit lumen; and
expanding the prosthetic valve device into contact with the inner wall of the conduit.

18. The method of claim 17 further comprising bioeroding a protective layer disposed on the valve support region of the expandable support structure.
